(19) United States
(12) Patent Application Publication
(10) Pub. No.: US 2023/0091003 A1
(43) Pub. Date: Mar. 23, 2023

(54) PHYSIOLOGICAL WELL-BEING AND POSITIONING MONITORING

(71) Applicant: TurningMode, LLC, Novelty, OH (US)

(72) Inventors: Jorge Zapata, Chagrin Falls, OH (US); Carlos Eduardo Vargas Silva, Antioquia (CO)

(73) Assignee: TurningMode, LLC, Novelty, OH (US)

(21) Appl. No.: 17/947,815

(22) Filed: Sep. 19, 2022

Related U.S. Application Data
(60) Provisional application No. 63/245,322, filed on Sep. 17, 2021.

(51) Int. Cl. A61B 5/11; A61B 5/01; A61B 5/00; A61B 5/08; A61B 5/024

(52) U.S. Cl. CPC A61B 5/1128; A61B 5/01; A61B 5/7275; A61B 5/1114; A61B 5/0816; A61B 5/024; A61B 5/0024; A61B 5/7425; A61B 5/746

(57) ABSTRACT

One or more techniques and/or systems are disclosed for providing for improved monitoring of an individual, wherein imaging data is received from a plurality of imaging sensors. The received imaging data is aggregated and patterns at targeted data segments of the aggregated imaging data are analyzed using one or more algorithms to determine one or more values. The one or more values determined from the analyzed patterns are classified to represent at least one of a physiological state determination or a positioning of the individual. Data corresponding to the classified one or more values is then output and displayed.

This application claims priority to U.S. Provisional Patent Application Ser. No. 63/245,322, entitled PHYSIOLOGICAL WELL-BEING AND POSITIONING MONITORING, filed Sep. 17, 2021, which is incorporated herein by reference.

### BACKGROUND

In some clinical and non-clinical settings, the constant monitoring of the well-being and positioning of patients or other subjects is necessary. In these settings, the use of a precise, intense wearable, or connecting patient monitoring system (PMS) or other monitoring device, may not be available, not desired, not possible, or not required. Some example patients may be admitted into emergency rooms, general hospitals, palliative care centers, or nursing homes. The patients might exhibit symptomology related to recovering from specific severity of psychological, psychiatric, or stress symptoms; or might be undergoing palliative, neo-natal, long inpatient, or other long-term care. Babies, toddlers, the elderly, or other at-home individuals, might also require constant well-being and positioning monitoring, while the use of wearable or connecting PMS or other devices is not practical, not possible, or not necessary.

As a result of not being able to use a well-being or monitoring device, having a skilled professional instead monitor an individual's well-being and positioning might be necessary or desirable. However, such human monitoring can be cost prohibitive, impractical, or not possible due to lack of availability or other impediments.

### SUMMARY

This Summary is provided to introduce a selection of concepts in a simplified form that are further described below in the Detailed Description. This Summary is not intended to identify key factors or essential features of the claimed subject matter, nor is it intended to be used to limit the scope of the claimed subject matter.

One or more techniques and systems described herein can be utilized for remote (e.g., non-contact) physiological well-being and positioning monitoring. For example, systems and methods of monitoring, described herein, can utilize a combination of sensors to actively monitor a patient's well-being and positioning remotely from a distance, without the use of direct contact sensors applied to a patient.

In one implementation for providing for improved monitoring of an individual, imaging data is received from a plurality of imaging sensors. The received imaging data is aggregated and patterns of targeted data segments of the aggregated imaging data are analyzed using one or more algorithms to determine one or more values. The one or more values determined from the analyzed patterns are classified to represent at least one of a physiological state determination or a positioning of the individual. Data corresponding to the classified one or more values is then output and displayed.

To the accomplishment of the foregoing and related ends, the following description and annexed drawings set forth certain illustrative aspects and implementations. These are indicative of but a few of the various ways in which one or more aspects may be employed. Other aspects, advantages and novel features of the disclosure will become apparent from the following detailed description when considered in conjunction with the annexed drawings.

### BRIEF DESCRIPTION OF THE DRAWINGS

FIG. 1 is an illustration of an example implementation of well-being monitoring.

FIG. 2 is another illustration of an example implementation of well-being monitoring.

FIG. 3 is a component diagram illustrating one implementation of one or more portions of one or more systems for performing well-being monitoring.

FIG. 4 illustrates an example implementation of a method for performing well-being monitoring operations.

FIG. 5A is top view of a component of an example implementation of well-being monitoring.

FIG. 5B is a perspective of a component of an example implementation of well-being monitoring.

FIG. 6 is a perspective view of an example implementation of well-being monitoring.

FIGS. 7A, 7B, and 7C are component diagram illustrating one implementation of well-being monitoring.

FIG. 8 is a block diagram of an example computing environment suitable for implementing various examples of well-being monitoring.

### DETAILED DESCRIPTION

The claimed subject matter is now described with reference to the drawings, wherein like reference numerals are generally used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, structures and devices are shown in block diagram form in order to facilitate describing the claimed subject matter.

There is a need for better decision support systems for caregivers; an alternative to current complex, expensive Patient Monitoring Systems (PMS) that unnecessarily limits patient mobility, and that are prone for care-giver's alarm fatigue. Alternately, patients may be just checked by nurses a few times during their visit rounds, sometimes hours apart between checks. Current remote tele-sitting services are a valuable patient care solution, but these services lack parameter assessment, and rely on a remote caregiver monitoring video, and sound feeds of multiple remote patient rooms.

In one aspect a real-time, no-contact, AI enabled, autonomous decision support system that continuously reads, and trends a target patient's condition can be devised. In this aspect, for example, such a system can utilize customizable status and alerts that may be integrated into traditional workstations and mobile platforms. The systems described herein can improve patient care, and has the potential to reduce a patient's adverse events when used in high Patient to Nurse (PTN) scenarios. In some implementations, the example systems can combine imaging hardware with AI trained to recognize target situations to provide customizable user interface experiences, including physiological conditions and patient movements. In this aspect, the systems and method described herein can provide a real-time, zero-contact, autonomous decision support system for caregivers. For example, the exemplary systems can provide real-time feedback about a subject's well-being based on multi-physiological parameters, subject motion and positioning change analysis using AI proprietary algorithms. The systems can continuously read, and trends parameters against an automatically, or user defined baselines.

The methods and systems disclosed herein, for example, may be suitable for use in myriad situations (e.g., hospital room, emergency room, nursing home room, patient bedroom, etc.) having one or more target individuals disposed therein to provide one or more monitoring features. For example, automated remote monitoring of various implementations utilizes one or more combinations and applications of imaging sensors to distinguish patterns of aggregated data from the imaging sensors in real-time to provide well-being and positioning monitoring information. It should be appreciated examples described herein can be used in different settings or environments in combination with various different types of imaging sensors. The examples given herein are merely for illustrative purposes.

In some implementations, the methods and systems described herein are able to detect and report a plurality of physiological and portion/motion conditions for a target patient. For example, the systems can detect the heart rate of a target patient, such as indicated by blood perfusion, which is rate at which blood is delivered to tissues, such as in the capillary beds. Further, breathing can be detected, as indicated by tidal respiration volume. Additionally, the motion of the target patient can be detected, such as ocular motion, global (e.g., overall) motion, motion of extremities, head motion, and foreign object motion in the target field. Positioning of the target patient may be detected, such as the subject elevation (e.g., height above the ground, bed, etc.), a global scene map of objects in the target field, and relation of the subject to the global map scene. Other physiological conditions may be monitored, such as patient temperature (e.g., forehead temperature, global temperature, distal temperature, ROI temperature), and other object (in the field) temperatures. These indications can be collected and provided in a user interface to a caregiver, and/or to an automated alert system, to help monitor one or more patients in real-time.

As an example, in some implementations, indications of blood perfusion, relating to heart rate monitoring, can provide early indication of conditions, and possible prevention. For example, skin changes, swelling, hot flashes, cognitive, fatigue; clammy, paling skin breakdown (pressure ulcers)/infection/necrosis may be indicated by blood perfusion monitoring. Further, internal bleeding, heart alterations, myocarditis, intervascular volume status, and stroke volume are other conditions that can be indicated by blood perfusion monitoring. Additionally, other useful indictors using blood perfusion can include, trauma patient single or multiple organ failure, for use with multiple patient observation at lower cost to triage change over time &/or stability; sepsis progression/organ function degradation leading to failure; single organ leading to systematic chain reaction; response to vasoconstriction regiment for central vs peripheral differential tied to vasoconstriction medication regiment; and perfusion change as an indicator to subsequent parameters issue advancement.

As another example, in some implementations, indications of tidal respiration, relating to breathing monitoring, can provide early indication of conditions, and possible prevention. For example, rapid breathing, slowing breathing, and erratic breathing are all indicative of downstream next issues. Other useful indicators can include pre & post intervention assessment with objective trending related to breathing, RSV, C19, asthmatic, and upper respiratory issues. Other tidal respiration data can indicate hallucination onset, seizure, stroke, cardiac risk; as well as psych Issues, such as anxiety/confusion/flight risk/self-harm. Additionally, tidal respiration may help indicate response to titrated analgesics, where, if apnea occurs, could lead to arrest; Further indications may include, over or under ventilation, and CO2 rise=downward trend.

As another example, subject motion monitoring can provide early indication and prevention of certain conditions. For example, ocular motion monitoring can detect a level of consciousness/fall risk, alertness; tumor presence or progressions, concussion, diabetic retinopathy, retinal detachment, seizures, hallucinations, macular deg, retinal edema, and seizure. Head motion and global monitoring can provide indication of seizures, level of consciousness, confusion, and stroke. Extremities motion monitoring can provide indications for a fall candidate, infusion tubing removal risk, TIA mini-strokes, seizures, possible respiratory distress, and a cardiac event. Further, foreign object motion monitoring can provide indications of a failure to interact with bed-side table successfully, perception/eyesight failure, in-room equipment failure, such as IV pole falling, bed rails being moved/covers moving erratically, Munchausen Syndrome-deliberate attempt to harm a patient; parents to kids; adult kids to parents; intentionally resetting alarms and/or pushing drugs into IVs. Additionally, motion monitoring may be able help with psychiatric ward monitoring, such as objective assessment of medication effectiveness, and aggressive trending, patient assessments.

As another example, patient temperature monitoring can provide early indication and prevention of certain conditions. For example, temperature monitoring can detect indications, where an increase could indicate an infection; viral or bacterial, heat exhaustion, inflammatory conditions (RA), malignant tumors; a decrease may indicate a loss of fat, changes in medication reaction and/or effect of meds they are on; a sudden change from range may indicate hypothermia; or systems are shutting down. A distal temperature monitory can provide indications of perfusion issues (see Perfusion), renal disease impact, diabetic conditions, and chronic infection susceptible, which may facilitate Hospice and family final moments caregiving. Further, ROI temperature monitoring can provide indications of IV infiltration, IV extravasation, changes in tissues; surgical site infection, tubing to tissue infection, incisions, scarring infection, and areas of trauma that may be degrading.

As another example, patient positioning monitoring can provide other indications of conditions that may be prevented or mitigated. For example, patient positioning monitoring may identify erratic movement such as thrashing, rapid change of positioning, decrease of motion compared to prior mobility. Other observations may include a reduction in motion or slowing changes, which may be equally valuable to alert a caregiver of a position change that could be leading to a fall, or a lack of position change that could be leading to a long-term tissue elevated pressure. Such a system may also infer a long-term, real-time pressure mapping of patients in bed, based on motion.

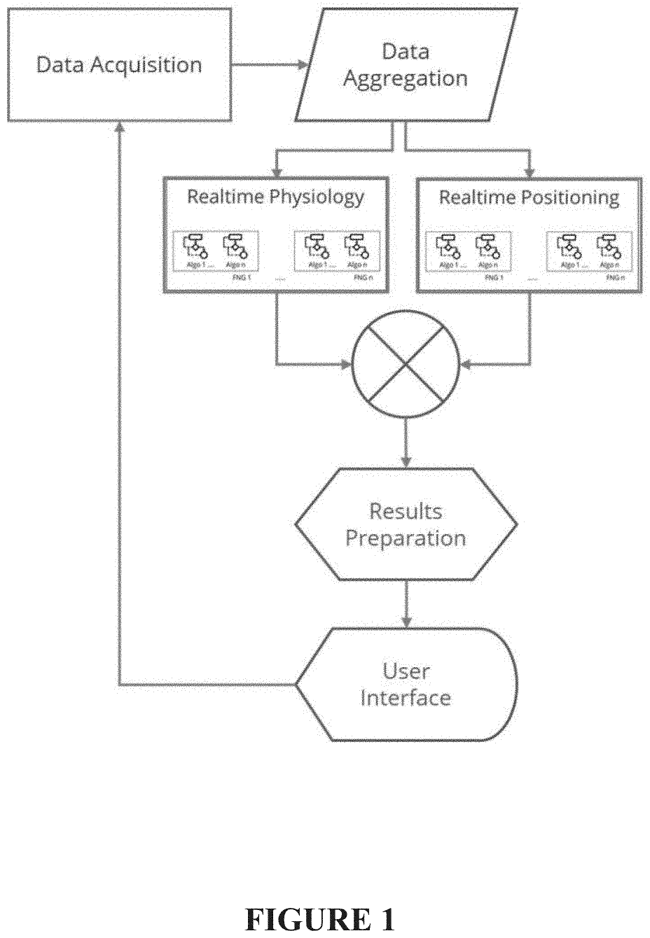

As one illustrative example, a depiction of a process 100 for physiological well-being and positioning monitoring is illustrated in FIG. 1. The process 100 is first initiated, such as by activating one or more monitoring features via a user device or interface, to enable the system. In response to activating the one or more monitoring features, data acquisition is performed at 102. For example, as described in more detail herein, one or more imaging sensors 120 acquire data of one or more objects within a room. One particular example includes one or more imaging sensors 120 configured to acquire images of a patient within a hospital room for well-being and positioning monitoring.

The acquired data is then aggregated at 104. For example, one or more data aggregation operations are performed to combine different data acquired by the one or more imaging sensors 120. It should be appreciated that the imaging sensors 120 can be located in different locations within the room and have different positions, orientations, field-of-views, etc., such as based on the type of images to be acquired, the processing to be performed on the acquired image data (also referred to as imaging data), etc. As such, different perspectives or views of the room or portions of the room can be acquired. In some implementations, the data aggregation is performed on different categories of acquired data. For example, as shown in FIG. 1, physiological and position data are separately aggregated into datasets and then real-time processing of each of the datasets is performed at 106a, 106b using one or more algorithms. In some implementations, when processing the datasets, an automatic or manual function sets biometrics/physiology parameters, and a different function sets boundaries and positioning parameters. That is, different parameters are used or set for processing each of the datasets.

In one implementation, the processing at 106a by a combination of the one or more algorithms analyzes recognized patterns at targeted data segments and classifies values against configurable thresholds to represent physiological state determinations. In one implementation, the processing at 106b by a combination of the one or more algorithms analyzes the recognized patterns at targeted data segments and classifies values against configurable areas of interest to represent the positioning of the patient.

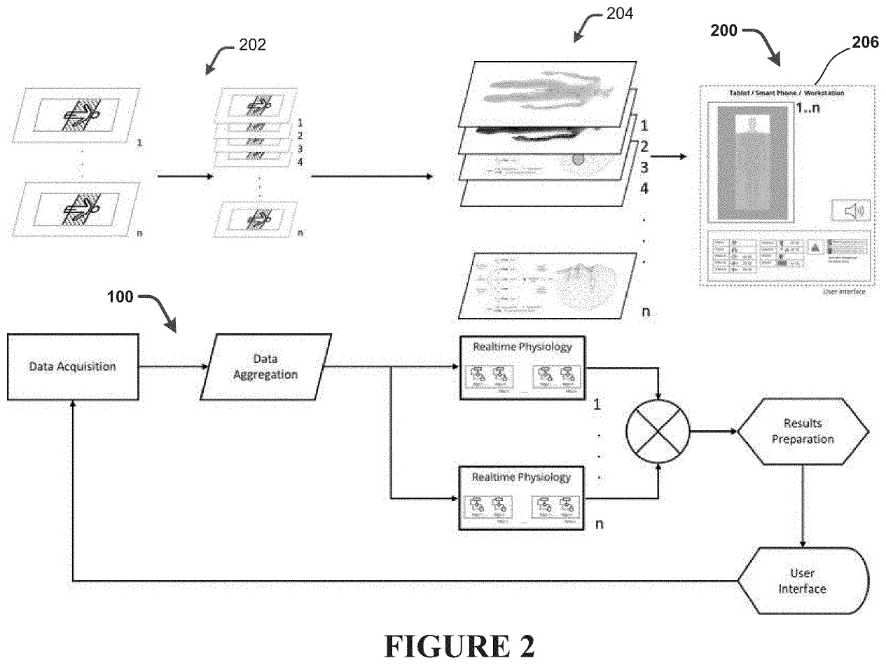

In some implementations, the physiological state determinations and the positioning of the patient results are then combined to enhance the available information and monitoring of the patient. For example, the output of the processing of the datasets by the one or more algorithms is then combined at 108 and results prepared at 110, such as for display at a user interface at 112. In one implementation, the output from the processing by the one or more algorithms is encrypted and the results are prepared by performing an automatic function that receives the encrypted algorithm results, decrypts the results, and organizes the results for display via a user interface, such as on an end-use device 200 (see FIG. 2). For example, the prepared results are output using one or more graphical and audio formats.

Thus, in one implementation, a combination of algorithms registers, aggregates, and combines data from at least two imaging sensors 120 to augment the usefulness of the resulting data set for analysis and subsequent inferences. That is, more effective well-being and positioning monitoring can be performed using the process 100. In some examples, the process 100 allows for real-time, remote, autonomous, patient physiological well-being and positioning monitoring.

In some implementations, a combination of algorithms harvests data from one or more data repositories for classification, curation, and augmentation, for the analysis thereof and subsequent inferences. For example, one or more datasets are acquired from stored data and used in the various implementations to perform one or more operations and/or enhance one or more operations.

As illustrated in FIG. 2, feedback from the user interface or one or more inputs received at the user interface can be used in subsequent processing, including in controlling the data acquisition. For example, the feedback or one or more inputs results in an adjustment of the type or amount of data acquired by the one or more imaging sensors 120, the settings of the one or more imaging sensors 120, the individual imaging sensors 120 selected to acquire the data, etc.

As can be seen in FIG. 2, different types of data 202 and outputs 204 can be generated using the process 100. For example, the one or more algorithms process the acquired image data to determine different physiological properties of the patient and different positioning information of the patient as represented by the data 204. And, as can be seen, a user interface 206 allows for the display of the different types of data, which can include images, text, sounds, etc. and can be formatted or displayed in different ways (e.g., charts, graphs, etc.).

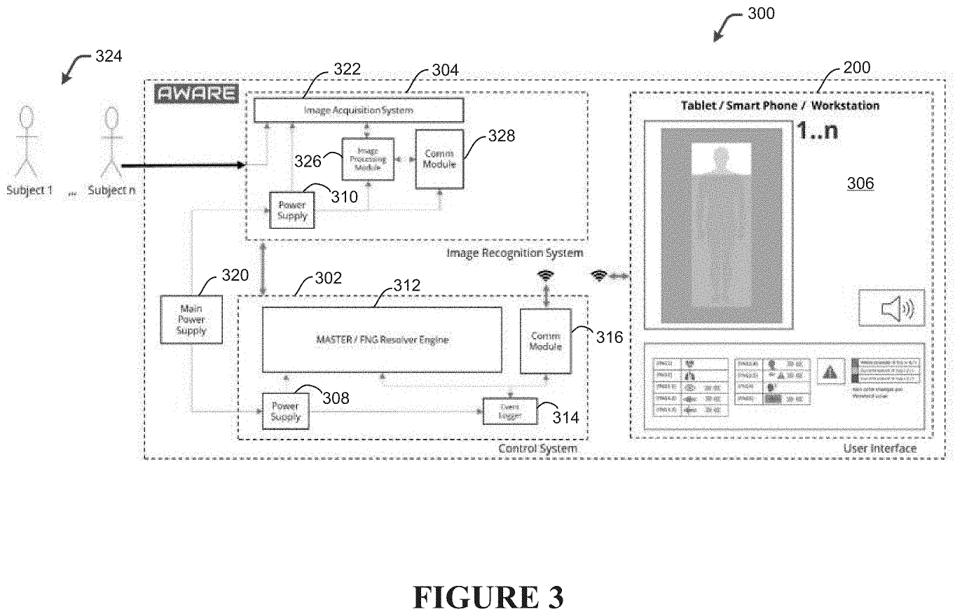

A system configuration of one implementation is illustrated in FIG. 3. In the example of this implementation, the system configuration is a monitoring system 300 that includes a control system 302, an image recognition system 304, and a user interface 306. It should be noted that one or more of the control system 302, the image recognition system 304, and the user interface 306 can be implemented in hardware, software, or a combination thereof.

In some examples, one or more of the control system 302, the image recognition system 304, and the user interface 306 are configured as sub-systems that together implement the process 100. In the illustrated example, the system 300 includes a main power supply 320 that routes power to a power supply 308 of the control system 302 and a power supply 310 of the image recognition system 304. For example, the power supplies 308, 310 can be local or “on-board” power supplies that power the components of each of the control system 302 and image recognition system 304.

In the illustrated implementation, the image recognition system 304 further includes an image acquisition system 322 that receives image data of one or more subjects 324 and an image processing module 326 that processes the received image data. For example, the image processing module 326 pre-processes or filters received images to be processed by the control system 302. In one example, the image processing module 326 is configured to perform segmentation or other object detection techniques on the received image data to identify objects or data of interest in the images. The image recognition system also includes a communication module 328 that allows for communication with, for example, the control system 302. Additionally, the image processing module 326 in some examples then analyzes the image data to determine well-being and/or positioning information as described in more detail herein. For example, the image processing module 326 uses the one or more algorithms to identify different properties or characteristics of the image data corresponding to a state or condition of the subject(s) 324.

The control system 302 further includes a resolver engine 312 and an event logger 314 connected to a communication module 316 (e.g., a wireless communication device). The control system 302 is configured to receive data from the image recognition system 304 and prepare the data for transmission and display on the end-user device 200. For example, processed and prepared data is wirelessly transmitted by the communication module 316 to a receiver (not shown) of the end-user device 200, which then displays the data via the user interface 306. For example, the resolver engine 312 organizes, filters, and/or sorts the processed data for transmission and display via the user interface 306. The event logger 314 tracks the data that is communicated to and from the control system 304.

In operation, the control system 302 in some examples is configured as a sub-system that organizes and encrypts the results of the algorithm processing described herein and transmits the results to the end-user device 200, such as a remote device (e.g., smartphone, tablet, or other workstation) for interfacing and/or interaction by a user with the user interface 306 (e.g., via display and sound interfacing). In one implementation, the end-user device 200 is configured (e.g., has an application installed thereon) that receives the encrypted results of the algorithm processing, decrypts the results, and organizes the results to one or multiple end-user using graphical and sound formats.

In some examples, the control system is configured as a sub-system that chronologically organizes the results of the algorithm processing (using the event logger 314) in text format and keeps an auditable, size-configurable first-in first-out (FIFO) log of results. The ordering in one example is based on a time-stamping of the processed image data.

Various examples are operable in different environments and for different applications, such as where well-being and patient positioning monitoring is needed while no wearable or connecting PMS is required, desired, or is viable, including, but not limited to:

1. Psychiatric patients, palliative patients, infant patients with available, but restricted range of motion, with the need or recommended well-being and/or positioning monitoring in which a wearable or connected PMS, or other monitoring device is detrimental to the patient's freedom of motion or is not practical, available, or necessary.

2. Emergency room patients during a triage stage with problematic or no access to PMS equipment, and with necessary and/or recommended well-being and/or positioning monitoring.

3. Patients or subjects in nursing homes and/or home care with available but restricted range of motion, with a need for or recommended well-being and positioning remote monitoring

4. Other subjects of a population, with available but restricted range of motion, with a need for or recommended well-being and positioning remote monitoring, threshold violation indications, and/or recording.

It should be appreciated that the problems overcome by one or more implementations of the present disclosure are applicable to patient monitoring at one or more different stages of the healthcare continuum, where patient well-being and/or patient positioning is necessary, but in which a connecting or wearable PMS or other device is not practical, not possible, or is not necessary.

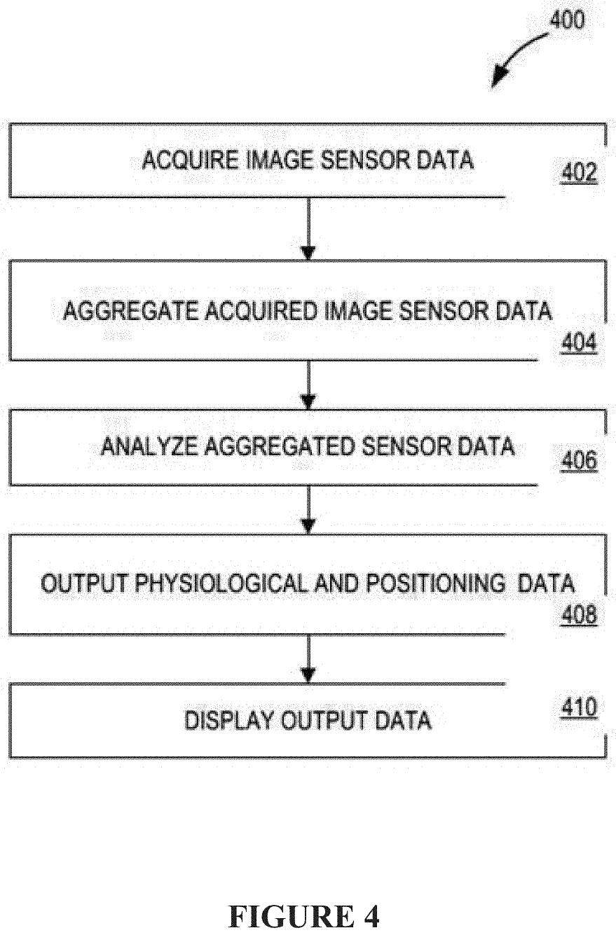

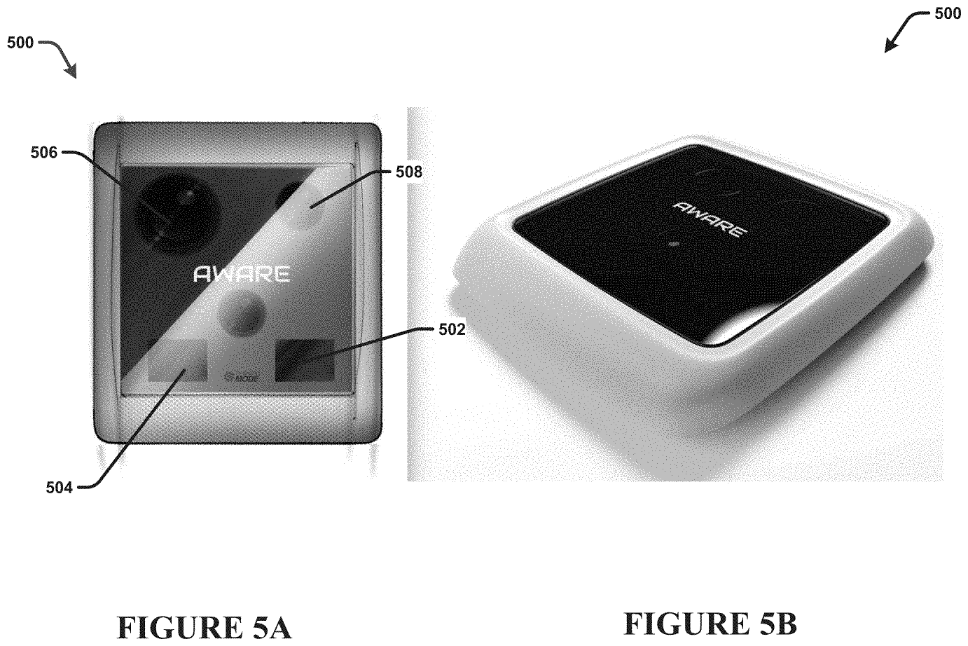

FIG. 4 is a flowchart 400 illustrating operations involved in physiological well-being and positioning monitoring according to one implementation. In some examples, the operations of flowchart 400 are performed by a computing device 500 illustrated in FIG. 5. The flowchart 400 commences with operation 402, which includes acquiring image sensor data. For example, one or more sensors (e.g., the imaging sensors 120) continuously image a space, such as a patient's room or a portion of a patient's room. In some examples, the acquired image sensor data is high-resolution image data (e.g., 4K image data), which can be a series of still images or video images. The image sensor data can be acquired at different resolutions, different angles, etc., such as based on the desired or required output. The one or more sensors in some implementations are any type of sensor (e.g., depth imaging sensor) capable of use to create a digital rendering of the room and objects therein being imaged (with data relating to other elements, such as objects in the room not relevant or related to the patient care (e.g., a table), capable of being removed from the data to be processed). For example, depth sensors can be used to set boundaries at defined depths (that can be changed) to capture desired image data (e.g., baby crib is set as a boundary, bed or floor set as boundary, etc.). It should be noted that the boundaries can be dynamically changed (e.g., parent can draw boundaries on a displayed image that is then used to set the imaging or processing space).

It should further be noted that the one or more sensors in various implementations are not diagnostic devices, but devices that only capture image data within the room. That is, the sensors do not directly acquire diagnostic data, but instead acquire image data that can be analyzed for monitoring purposes, and to support a caregiver's decision-making process as described in more detail herein. As a result, the various examples are useful in more applications, including applications where monitoring sensors cannot or are not desired to be used.

At operation 404, the acquired image data is aggregated. For example, the image data from a set of cameras within a room is combined into a larger image dataset that is stored for analysis. In some examples, the aggregated data includes only data relating to monitoring activity for the patient as described in more detail herein. In one example, the image data is segmented or filtered to include data (e.g., image pixel data) of the patient and the immediately surrounding area (e.g., patient bed).

At operation 406, the aggregated image data is analyzed to determine, for example, physiological and/or positioning data for the monitored patient. For example, using one or more algorithms as described herein, the image data is processed to determine the physiological and/or positioning data. For example, one or more different image processing techniques can be used to determine respiration, blood perfusion, heart rate, and ocular motion (or other bio-signals, such as temperature, brain activity, etc.), among others. In one example, the imaging data from the different imaging sensors is processed to detect pixelation, such as in the eyes, ears, cheeks, etc. of the patient. In some examples, the processing is performed on image data within a defined spectrum, such as the infrared (IR) spectrum (e.g., 350 nm-750 nm light spectrum). It should be noted that in various examples, calibration is automatically set, such as to distinguish between the background and the person (e.g., the patient).

As another example, a light sensor is used wherein an IR grid of dots is transmitted or projected and one or more image cameras “read” the dots. The changing geometry of dots and different depths can be used in the analysis to identify the different patient properties, states, etc.

As still another example, when imaging portions of the skin that are thin, the image data is analyzed to determine changes (e.g., delta changes) of the skin between image frames. Using a correlation (e.g., 1:1), changes in the coloration are representative of changes in blood perfusion.

As yet another example, changes in pixelation between the nose and mouth and changes in temperature can be analyzed to determine respiration, including tidal volume over time.

In some examples, the analysis performed at 406 is based on or uses machine learning. For example, online laboratory simulations can be performed to improve the analysis for different desired properties. It should be noted that any machine learning techniques can be used.

Physiological and/or positioning data is output at 408 and displayed at 410. For example, colored respiration data or heart rate data can be displayed, with the coloring determined based on one or more thresholds. The output data can be representative of different states of the patient, for example, an awakeness of the patient (e.g., whether patient is awake or asleep, in distress, etc.). The data can then be used to proactively support the decision on patient care and can be displayed on different devices, in real time, for that purpose (e.g., a visual on a mobile phone).

Thus, one or more implementations allow for proactive, touchless communication, for example, to a nurse or other caregiver responsible for the care of the patient. The data or information provided to the nurse or other caregiver can be used to make decisions or set different parameters. For example, a “geofence” can be set to determine movement beyond a visual (on the mobile phone) barrier. That is, a monitored area can be set. As another example, patient movement can be tracked, such as very small or minute motion (e.g. to 200 milliseconds (ms)) to identify such movement for patient treatment (such as during surgery) or for imaging use.

It should be noted that the implementations described herein can be used in different environments, such as a hospital, nursing home, rehabilitation center, etc. Additionally, the herein described implementations can be used to monitor individuals of any age (e.g., babies, infants, elderly) or non-humans (e.g., animals). In some examples, the imaging devices include processing capabilities to perform one or more operations described herein. As such, applications specific sensors are configured in some examples, such as a nursing sensor or a baby monitor. In one example, a monitor for delta variant changes can be used, such as for cyber-knife treatment for cancer wherein real-time updates to the movement of the patient can be used to precisely apply radiation to a treatment site. In some examples, azimuths of the imaging space are updated within the system to adjust the imaging, such that no calibration or human interactions are needed.

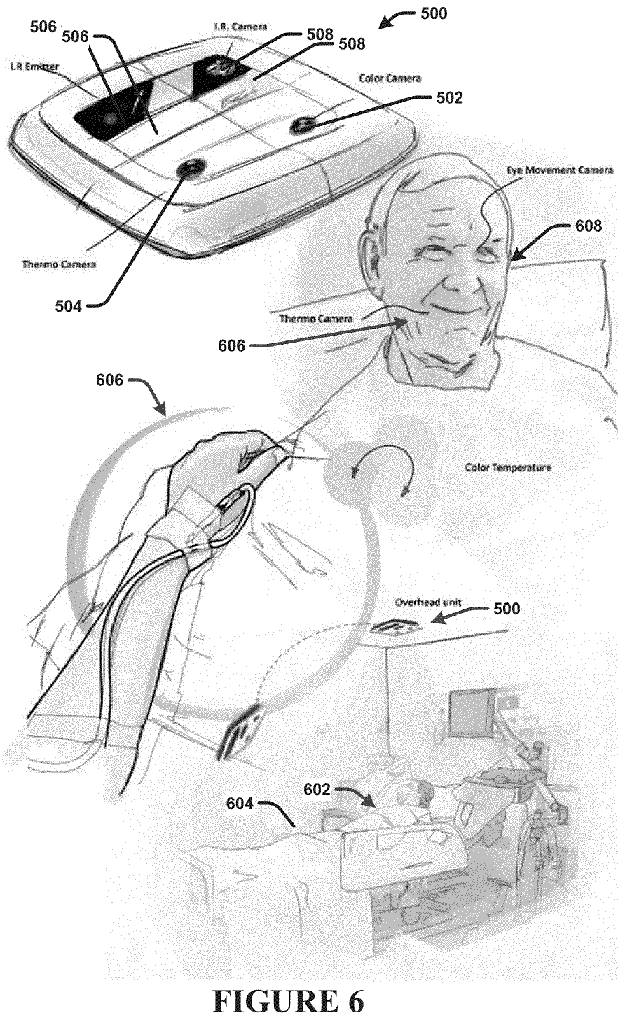

FIGS. 5A and 5B illustrate an example implementation of a patient monitoring device 500 that comprises one or more portions of the systems described herein. FIG. 6 illustrates an example implementation of the example device 500. In some implementations, the example device 500 can be disposed above (e.g., or otherwise in proximity to) a target patient, whom may be situated in a target area 604 (e.g., field of view). The patient monitoring device 500 may be positioned anywhere chosen with sound engineering judgment to receive data from the patient. In one implementation, it may be positioned on a ceiling. In another implementation, it may be positioned directly overhead of the patient. In yet another implementation, it may be located on a wall. In some implementations, monitoring device 500 may be disposed on a stationary or movable bracket to change the angle of the element 500 relative to the patient so that element 500 may receive data input and transmit it for processing using process 100. In this example implementation, the patient monitoring device 500 can comprise a video camera 502 that is configured to capture video images of the target area 604 in color. As an example, the video camera can detect motion 608 is the target area, which may help identify certain conditions and patient movements, as described above.

Further, the patient monitoring device 500 can comprise a thermal camera 504 that is configured to capture thermal imagery 606 of the target area 604, such as one that detects temperatures and generates a thermal map that distinguishes different temperatures by different colors. In this example, the thermal imagery 606 can distinguish temperature by colors to create a thermal map, and to detect potential anomalies, and or certain conditions, as described above. The patient monitoring device 500 can also comprise an infrared light emitter 506, that can be used in conjunction with an infrared camera 508. The infrared light emitter 506 can produce infrared light that can reflect off objects in the target area 604, which may be detected by the infrared camera 508. This can allow for image capture in low (visible) light situations.

In this implementation, the example device 500 can be configured to be readily modular, such that it can be readily moved from a first location to a second, desired location easily. Communication between the device and a remote computer (e.g., workstation, laptop, tablet, handheld, etc.) can be through wireless protocols, such as Wi-Fi, cellular, near-field, Bluetooth, etc. In some implementation, a wired solution can be provided that allows for coupling the device to communication cables (e.g., Cat 5/10) for communicating with a base station, and/or a modem for wireless communication.

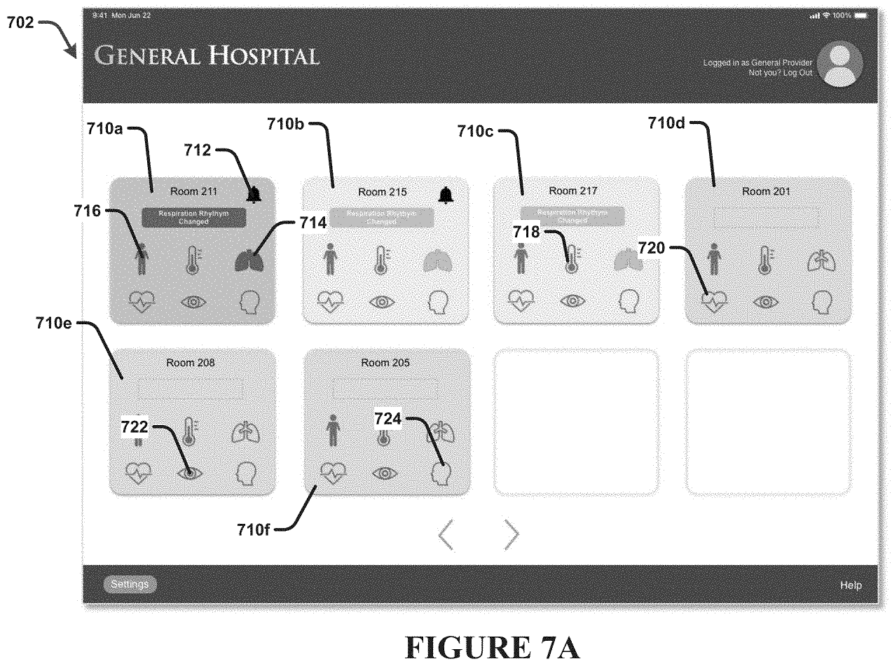

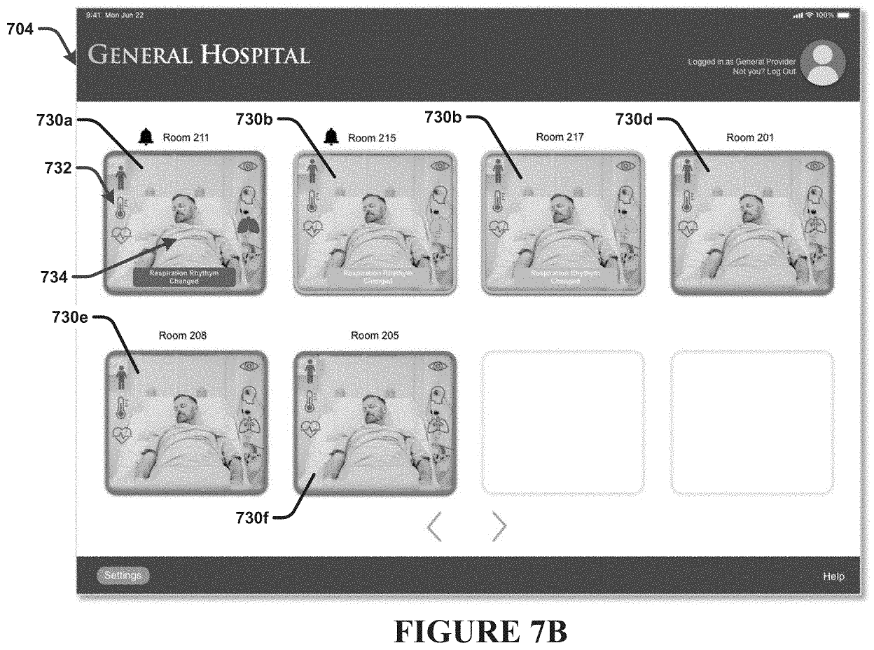

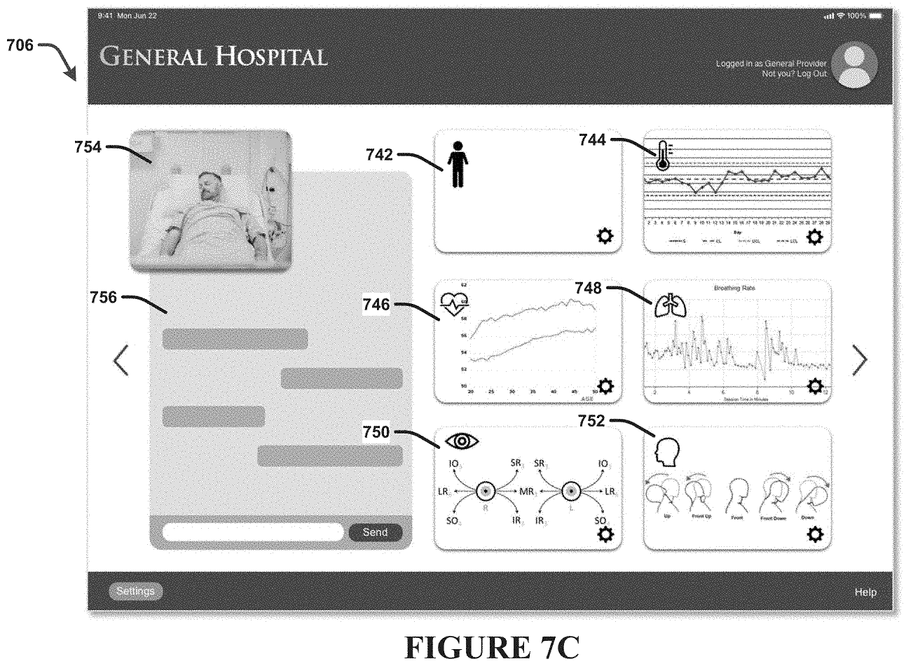

FIGS. 7A, 7B, and 7C illustrate example user interface (UI) implementations that utilize one or more portions of one or more systems and methods described herein. As one example, the UI(s) can be implemented on any suitable display, including, but not limited to, a workstation display, computer monitor, tablet, handheld, and others. The UI display 702 indicates an alert status modules 710a-f for multiple target patients, for example, in different rooms listed by room number. In this example, the alert module 710 can provide various levels of alerts for various conditions, as described herein. As one example, module 710a indicates an alert 712 that indicates a respiratory change, highlighting the alert in red, and highlighting the lungs symbol 714 to indicate respiration. Other symbols can include the motion (person) symbol 716, temperature symbol 718, heart symbol 720, eye movement symbol 722, and head movement symbol 724—others are anticipated. As another example, modules 710b and c, indicate respiratory changes that may be less serious, highlighted in yellow. UI modules 710d-f indicate normal, or at least within threshold limits, for the target patients, which are highlighted in green. In some implementations, the levels of alerts can be automatically set, and/or set manually by an operator. It is anticipated that various indicators can be used, and the pictures, colors and other indicators may be used as desired by the operators.

As another example, in FIG. 7B, the various UI modules 730a-f are indicative of the similar patient rooms, for each patient. In this example the same symbols 732 (or different) can be utilized to provide for an alert status. Further, as illustrated, UI module 730a indicates a red highlighted alert status for that patient, indicative of a change in respiration. Further, in this example, a video image 734 of the respective patients. In some implementations, that can be a live feed that show real-time images of the patient, in-line with the other alert indicators and symbols.

As another example, in FIG. 7C, an individual target patient UI module 706 is illustrated. In this example, the various details of each monitoring indicator is provided. Here, target patient movement 742 is detailed, patient temperature 744, heart rate 746, respiration 748, eye movement 750, and head movement 752 are detailed. As an example, an operator (e.g., caregiver) may select an individual UI module (e.g., 710, 730) to drill down on the specific details for the selected target patient. Further, in this example, a video image 754 can be provided that indicates a real-time image of the target patient. Additionally, a patient's chart 756 can be displayed that indicates medical information and instructions for the target patient.

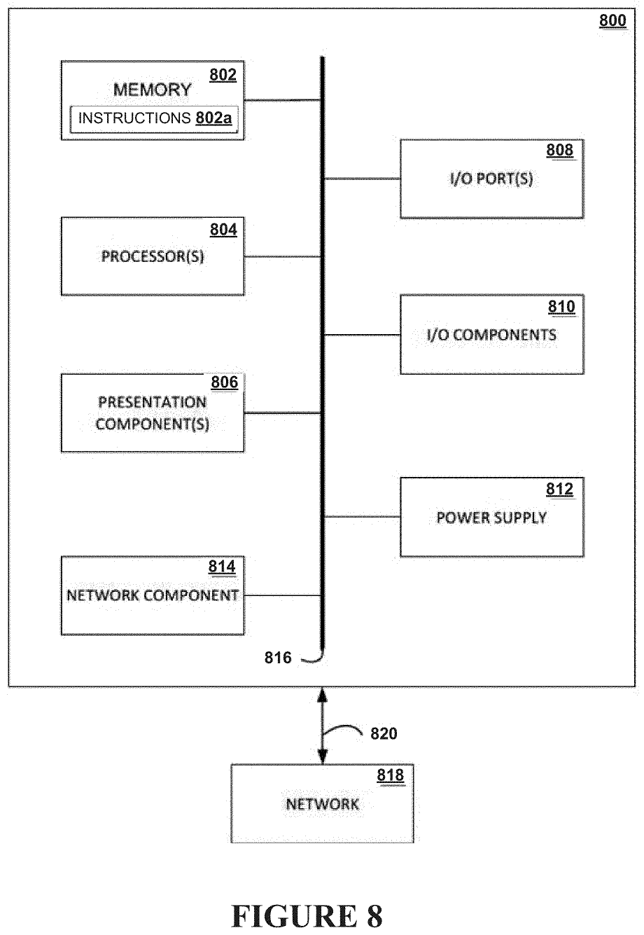

With reference now to FIG. 8, a block diagram of the computing device 800 suitable for implementing various aspects of the disclosure is described (e.g., a monitoring system). FIG. 8 and the following discussion provide a brief, general description of a computing environment in/on which one or more or the implementations of one or more of the methods and/or system set forth herein may be implemented. The operating environment of FIG. 8 is merely an example of a suitable operating environment and is not intended to suggest any limitation as to the scope of use or functionality of the operating environment. Example computing devices include, but are not limited to, personal computers, server computers, hand-held or laptop devices, mobile devices (such as mobile phones, mobile consoles, tablets, media players, and the like), multiprocessor systems, consumer electronics, mini computers, mainframe computers, distributed computing environments that include any of the above systems or devices, and the like.

Although not required, implementations are described in the general context of “computer readable instructions” executed by one or more computing devices. Computer readable instructions may be distributed via computer readable media (discussed below). Computer readable instructions may be implemented as program modules, such as functions, objects, Application Programming Interfaces (APIs), data structures, and the like, that perform particular tasks or implement particular abstract data types. Typically, the functionality of the computer readable instructions may be combined or distributed as desired in various environments.

In some examples, the computing device 800 includes a memory 802, one or more processors 804, and one or more presentation components 806. The disclosed examples associated with the computing device 800 are practiced by a variety of computing devices, including personal computers, laptops, smart phones, mobile tablets, hand-held devices, consumer electronics, specialty computing devices, etc. Distinction is not made between such categories as “workstation,” “server,” “laptop,” “hand-held device,” etc., as all are contemplated within the scope of FIG. 8 and the references herein to a “computing device.” The disclosed examples are also practiced in distributed computing environments, where tasks are performed by remote-processing devices that are linked through a communications network. Further, while the computing device 800 is depicted as a single device, in one example, multiple computing devices work together and share the depicted device resources. For instance, in one example, the memory 802 is distributed across multiple devices, the processor(s) 804 provided are housed on different devices, and so on.

In one example, the memory 802 includes any of the computer-readable media discussed herein. In one example, the memory 802 is used to store and access instructions 802a configured to carry out the various operations disclosed herein. In some examples, the memory 802 includes computer storage media in the form of volatile and/or nonvolatile memory, removable or non-removable memory, data disks in virtual environments, or a combination thereof. In one example, the processor(s) 804 includes any quantity of processing units that read data from various entities, such as the memory 802 or input/output (I/O) components 810. Specifically, the processor(s) 804 are programmed to execute computer-executable instructions for implementing aspects of the disclosure. In one example, the instructions 802a are performed by the processor 804, by multiple processors within the computing device 800, or by a processor external to the computing device 800. In some examples, the processor(s) 804 are programmed to execute instructions such as those illustrated in the flow charts discussed herein and depicted in the accompanying drawings.

In other implementations, the computing device 800 may include additional features and/or functionality. For example, the computing device 800 may also include additional storage (e.g., removable and/or non-removable) including, but not limited to, magnetic storage, optical storage, and the like. Such additional storage is illustrated in FIG. 8 by the memory 802. In one implementation, computer readable instructions to implement one or more implementations provided herein may be in the memory 802 as described herein. The memory 802 may also store other computer readable instructions to implement an operating system, an application program and the like. Computer readable instructions may be loaded in the memory 802 for execution by the processor(s) 804, for example.

The presentation component(s) 806 present data indications to an operator or to another device. In one example, the presentation components 806 include a display device, speaker, printing component, vibrating component, etc. One skilled in the art will understand and appreciate that computer data is presented in a number of ways, such as visually in a graphical user interface (GUI), audibly through speakers, wirelessly between the computing device 800, across a wired connection, or in other ways. In one example, the presentation component(s) 806 are not used when processes and operations are sufficiently automated that a need for human interaction is lessened or not needed. I/O ports 808 allow the computing device 800 to be logically coupled to other devices including the I/O components 810, some of which is built in. Implementations of the I/O components 810 include, for example but without limitation, a microphone, keyboard, mouse, joystick, pen, game pad, satellite dish, scanner, printer, wireless device, camera, etc.

The computing device 800 includes a bus 816 that directly or indirectly couples the following devices: the memory 802, the one or more processors 804, the one or more presentation components 806, the input/output (I/O) ports 808, the I/O components 810, a power supply 812, and a network component 814. The computing device 800 should not be interpreted as having any dependency or requirement related to any single component or combination of components illustrated therein. The bus 816 represents one or more busses (such as an address bus, data bus, or a combination thereof). Although the various blocks of FIG. 8 are shown with lines for the sake of clarity, some implementations blur functionality over various different components described herein.

The components of the computing device 800 may be connected by various interconnects. Such interconnects may include a Peripheral Component Interconnect (PCI), such as PCI Express, a Universal Serial Bus (USB), firewire (IEEE 1394), an optical bus structure, and the like. In another implementation, components of the computing device 800 may be interconnected by a network. For example, the memory 802 may be comprised of multiple physical memory units located in different physical locations interconnected by a network.

In some examples, the computing device 800 is communicatively coupled to a network 818 using the network component 814. In some examples, the network component 814 includes a network interface card and/or computer-executable instructions (e.g., a driver) for operating the network interface card. In one example, communication between the computing device 800 and other devices occurs using any protocol or mechanism over a wired or wireless connection 820. In some examples, the network component 814 is operable to communicate data over public, private, or hybrid (public and private) connections using a transfer protocol, between devices wirelessly using short range communication technologies (e.g., near-field communication (NFC), Bluetooth® branded communications, or the like), or a combination thereof.

The connection 820 may include, but is not limited to, a modem, a Network Interface Card (NIC), an integrated network interface, a radio frequency transmitter/receiver, an infrared port, a USB connection or other interfaces for connecting the computing device 800 to other computing devices. The connection 820 may transmit and/or receive communication media.

Although described in connection with the computing device 700, examples of the disclosure are capable of implementation with numerous other general-purpose or special-purpose computing system environments, configurations, or devices. Implementations of well-known computing systems, environments, and/or configurations that are suitable for use with aspects of the disclosure include, but are not limited to, smart phones, mobile tablets, mobile computing devices, personal computers, server computers, hand-held or laptop devices, multiprocessor systems, gaming consoles, microprocessor-based systems, set top boxes, programmable consumer electronics, mobile telephones, mobile computing and/or communication devices in wearable or accessory form factors (e.g., watches, glasses, headsets, or earphones), network PCs, minicomputers, mainframe computers, distributed computing environments that include any of the above systems or devices, VR devices, holographic device, and the like. Such systems or devices accept input from the user in any way, including from input devices such as a keyboard or pointing device, via gesture input, proximity input (such as by hovering), and/or via voice input.

Implementations of the disclosure are described in the general context of computer-executable instructions, such as program modules, executed by one or more computers or other devices in software, firmware, hardware, or a combination thereof. In one example, the computer-executable instructions are organized into one or more computer-executable components or modules. Generally, program modules include, but are not limited to, routines, programs, objects, components, and data structures that perform particular tasks or implement particular abstract data types. In one example, aspects of the disclosure are implemented with any number and organization of such components or modules. For example, aspects of the disclosure are not limited to the specific computer-executable instructions or the specific components or modules illustrated in the figures and described herein. Other examples of the disclosure include different computer-executable instructions or components having more or less functionality than illustrated and described herein. In implementations involving a general-purpose computer, aspects of the disclosure transform the general-purpose computer into a special-purpose computing device when configured to execute the instructions described herein.

By way of example and not limitation, computer readable media comprises computer storage media and communication media. Computer storage media include volatile and nonvolatile, removable, and non-removable memory implemented in any method or technology for storage of information such as computer readable instructions, data structures, program modules, or the like. Computer storage media are tangible and mutually exclusive to communication media. Computer storage media are implemented in hardware and exclude carrier waves and propagated signals. Computer storage media for purposes of this disclosure are not signals per se. In one example, computer storage media include hard disks, flash drives, solid-state memory, phase change random-access memory (PRAM), static random-access memory (SRAM), dynamic random-access memory (DRAM), other types of random-access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transmission medium used to store information for access by a computing device. In contrast, communication media typically embody computer readable instructions, data structures, program modules, or the like in a modulated data signal such as a carrier wave or other transport mechanism and include any information delivery media.

While various spatial and directional terms, including but not limited to top, bottom, lower, mid, lateral, horizontal, vertical, front and the like are used to describe the present disclosure, it is understood that such terms are merely used with respect to the orientations shown in the drawings. The orientations can be inverted, rotated, or otherwise changed, such that an upper portion is a lower portion, and vice versa, horizontal becomes vertical, and the like.

The word “exemplary” is used herein to mean serving as an example, instance or illustration. Any aspect or design described herein as “exemplary” is not necessarily to be construed as advantageous over other aspects or designs. Rather, use of the word exemplary is intended to present concepts in a concrete fashion. As used in this application, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, at least one of A and B and/or the like generally means A or B or both A and B. In addition, the articles “a” and “an” as used in this application and the appended claims may generally be construed to mean “one or more” unless specified otherwise or clear from context to be directed to a singular form.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims. Of course, those skilled in the art will recognize many modifications may be made to this configuration without departing from the scope or spirit of the claimed subject matter.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein.

Various operations of implementations are provided herein. In one implementation, one or more of the operations described may constitute computer readable instructions stored on one or more computer readable media, which if executed by a computing device, will cause the computing device to perform the operations described. The order in which some or all of the operations are described should not be construed as to imply that these operations are necessarily order dependent. Alternative ordering will be appreciated by one skilled in the art having the benefit of this description. Further, it will be understood that not all operations are necessarily present in each implementation provided herein.

Any range or value given herein can be extended or altered without losing the effect sought, as will be apparent to the skilled person.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure which performs the function in the herein illustrated exemplary implementations of the disclosure.

As used in this application, the terms “component,” “module,” “system,” “interface,” and the like are generally intended to refer to a computer-related entity, either hardware, a combination of hardware and software, software, or software in execution. For example, a component may be, but is not limited to being, a process running on a processor, a processor, an object, an executable, a thread of execution, a program and/or a computer. By way of illustration, both an application running on a controller and the controller can be a component. One or more components may reside within a process and/or thread of execution and a component may be localized on one computer and/or distributed between two or more computers.

Furthermore, the claimed subject matter may be implemented as a method, apparatus or article of manufacture using standard programming and/or engineering techniques to produce software, firmware, hardware or any combination thereof to control a computer to implement the disclosed subject matter. The term “article of manufacture” as used herein is intended to encompass a computer program accessible from any computer-readable device, carrier or media. Of course, those skilled in the art will recognize many modifications may be made to this configuration without departing from the scope or spirit of the claimed subject matter.

In addition, while a particular feature of the disclosure may have been disclosed with respect to only one of several implementations, such feature may be combined with one or more other features of the other implementations as may be desired and advantageous for any given or particular application. Furthermore, to the extent that the terms “includes,” “having,” “has,” “with,” or variants thereof are used in either the detailed description or the claims, such terms are intended to be inclusive in a manner similar to the term “comprising.”

The implementations have been described, hereinabove. It will be apparent to those skilled in the art that the above methods and apparatuses may incorporate changes and modifications without departing from the general scope of this invention. It is intended to include all such modifications and alterations in so far as they come within the scope of the appended claims or the equivalents thereof.

What is claimed is:

1. A system for remotely monitoring an individual, the system comprising:
a plurality of imaging sensors capturing images of a target individual;
a processor; and
a computer-readable medium storing instructions that are operative upon execution by the processor to:
receive imaging data from the plurality of imaging sensors;
aggregate the received imaging data;
analyze patterns at targeted data segments of the aggregated imaging data using one or more algorithms to determine one or more values;
classify the one or more values determined from the analyzed patterns to represent at least one of a physiological state determination or a positioning of the individual;
output data corresponding to the classified one or more values; and
display the output data on a display.

2. The system of claim 1, wherein the values are classified against configurable thresholds to represent the physiological state determination.

3. The system of claim 1, wherein the values are classified against configurable areas of interest to represent the positioning of the individual.

4. The system of claim 1, the plurality of imaging sensors comprising one or more of:
a video capture camera;
an infrared camera;
an infrared emitter; and
a thermal camera.

5. The system of claim 1, comprising a housing that houses the plurality of imaging sensors.

6. The system of claim 5, the processor and computer readable medium disposed in the housing, and performing the receiving, aggregating, analyzing, classifying, and outputting the data corresponding to the classified one or more values.

7. The system of claim 6, comprising a display that is configured to display a user interface (UI) indicative of the output data corresponding to the classified one or more values.

8. The system of claim 5, wherein the housing is portable such that it can be readily moved from a first location to a second location.

9. The system of claim 1, the plurality of imaging sensors capturing images of a target individual configured to capture one or more of the following for a target patient:
respiration;
heart rate;
temperature;
eye movement;
head movement; and
body movement.

10. The system of claim 9, wherein:
respiration is indicated by tidal respiration volume;
heart rate is indicated by blood perfusion; and
temperature is indicated by forehead temperature, distal temperature, global temperature, and/or ROI temperature.

11. The system of claim 1, comprising a display that is configured to display a user interface (UI) indicative of one or more physiological conditions of a target patient, and a motion condition of the target patient.

12. The system of claim 11, the UT comprising indicators of one or more of the following for a target patient:
respiration condition;
temperature condition;
position;
movement;
real-time images;
heart rate condition; and
medical background and instructions.

13. A system for remotely monitoring a target individual, comprising:
a housing;
a plurality of image sensors disposed in the housing, the plurality of image sensors configured to capture images of the target individual;
memory disposed in the housing, the memory storing instructions for processing the captured images into classified values indicative of a physiological status and/or position of the target individual; and
a processor disposed in the housing, the processor configured to process the stored instructions and received data indicative of the captured images;
wherein the classified values are transmitted to a remote display in such a condition as to allow the remote display to display the physiological status and/or position of the target individual in a user interface (UI); and
wherein the classified values are indicative of analyzed patterns of targeted data segments derived from the captured images sing one or more algorithms.

14. The system of claim 13, wherein the classified values are classified against configurable thresholds to represent the physiological state determination.

15. The system of claim 13, wherein the values are classified against configurable areas of interest to represent the positioning of the individual.

16. The system of claim 13, wherein the plurality of imaging sensors comprise one or more of:
a video capture camera;
an infrared camera;
an infrared emitter; and
a thermal camera.

17. The system of claim 13, the plurality of imaging sensors capturing images of a target individual configured to capture one or more of the following for a target patient:
respiration;
heart rate;
temperature;
eye movement;
head movement; and
body movement.

18. The system of claim 17, wherein:
respiration is indicated by tidal respiration volume;
heart rate is indicated by blood perfusion; and
temperature is indicated by forehead temperature, distal temperature, global temperature, and/or ROI temperature.

19. The system of claim 13, the UI comprising indicators of one or more of the following for a target patient:
respiration condition;
temperature condition;
position;
movement;
real-time images;
heart rate condition; and
medical background and instructions.

20. A method for remotely monitoring an individual using a system comprising a plurality of imaging sensors capturing images of a target individual, a processor, and computer-readable medium storing instructions that are operative upon execution by the processor, the method comprising:
using the plurality of image sensors to capture image data indicative of physiological conditions of a target individual;
aggregating the received imaging data;
analyzing patterns at targeted data segments of the aggregated imaging data using one or more algorithms to determine one or more values;
classifying the one or more values determined from the analyzed patterns to represent at least one of a physiological state determination or a positioning of the individual;
outputting display data corresponding to the classified one or more values; and
displaying the display data in the form of a user interface (UI) on a remote display.
